(19) United States
(12) Patent Application Publication
(10) Pub. No.: US 2014/0142092 A1
(43) Pub. Date: May 22, 2014

(54) COMPOUNDS AND METHODS FOR SKIN REPAIR

(71) Applicant: Allergan, Inc., Irvine, CA (US)

(72) Inventors: Robert M. Burk, Laguna Beach, CA (US); Wha-Bin Im, Irvine, CA (US)

(73) Assignee: Allergan, Inc., Irvine, CA (US)

(21) Appl. No.: 14/078,847

(22) Filed: Nov. 13, 2013

Related U.S. Application Data
(60) Provisional application No. 61/727,574, filed on Nov. 16, 2012.

(51) Int. Cl. A61K 31/5415; A61K 31/41; A61K 31/435; A61K 8/49; A61K 8/368; A61K 31/192; A61Q 19/08

(52) U.S. Cl. CPC A61K 31/5415; A61K 31/192; A61K 31/41; A61Q 19/08; A61K 8/49; A61K 8/494; A61K 8/368; A61K 31/435; A61K 8/4906
USPC 514/224.2; 514/568; 514/381; 514/345

(57) ABSTRACT

The disclosure provides compositions and methods for wound healing and scar reduction. The compositions and methods of the invention include at least one mixed EP2/EP4 agonist set forth herein. Wounds and or scars that can be treated by the compositions and methods of the invention can arise from events such as surgery, trauma, disease, mechanical injury, burn, radiation, poisoning, and the like.

### CROSS REFERENCE TO RELATED APPLICATIONS

This application claims the benefit of U.S. Provisional Application Ser. No. 61/727,574 filed on Nov. 16, 2012, which is incorporated by reference herein in its entirety.

### FIELD OF THE INVENTION

The invention relates generally to compositions and methods for wound healing, and particularly to the use of mixed EP₂/EP₄ agonists for treatment in wound healing, scar reduction, scar prevention and skin repair.

### BACKGROUND OF THE INVENTION

Prostanoid EP₄ receptor is a G protein-coupled receptor that mediates the actions of prostaglandin E₂ (PGE₂) and is characterized by the longest intracellular C terminus loop when compared to other prostanoid receptors. Mainly, EP₄ receptors couple to Gs and mediate elevations in cAMP concentration, although they do participate in other pathways as well. There are some redundancies in function between EP₂ and EP₄ receptors. For example, both receptors induce PGE₂-mediated RANKL through cAMP. However, EP₂ is involved in cumulus expansion in ovulation and fertilization, whereas EP₄ regulates closure of the ductus arteriosus. Expression of EP₄ receptors is controlled by various physiological and pathophysiological processes as these receptors participate in ovulation and fertilization, induce bone formation, protect against inflammatory bowel disease, facilitate Langerhans cell migration and maturation and mediate joint inflammation in a model of collagen-induced arthritis, among others

Skin blemishes such as flesh wounds, scars and wrinkles can occur on any area of the body. Scarring may occur in all parts of adult body, following local or systemic traumas such as mechanical injury, surgery, burn, radiation and poisoning, and represents a failure of homeostatic processes to restore normal structure at the wound sites. Wrinkles occur for a variety of reasons and are a common sign of aging. Both scars and signs of aging can typically considered undesirable.

Accordingly, an agent that safely and effectively treats or prevents such skin blemishes is highly desirable.

### SUMMARY OF THE INVENTION

The disclosure provides compositions and methods for wound healing, scar reduction, scar prevention and wrinkle treatment and prevention. The compositions and methods of the invention include at least one mixed EP₂/EP₄ agonist set forth herein. Wounds and or scars that can be treated by the compositions and methods of the invention can arise from events such as surgery, trauma, disease, mechanical injury, burn, radiation, poisoning, and the like.

In one embodiment of the invention there are provided methods of treating a skin wound or scar. Such methods are performed, for example, by administering to a subject in need thereof a composition comprising a therapeutically effective amount of a compound having a structure:

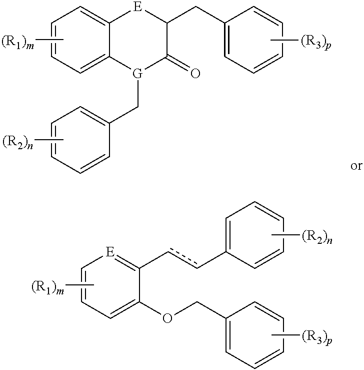

wherein:

R₁, R₂, and R₃ are each independently alkyl, alkoxy, halogen, trifluoromethyl, or —CO₂R₄, wherein R₄ is —H, C₁-C₆ alkyl, hydroxyalkyl, or

—CH₂CH₂OH; or

each R₂ is independently

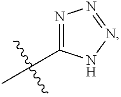 [SMILES: *c1nnn[nH]1]

acylsulfonamide, phosphonate, or —SO₃H;

each E is independently C, N, S or O;

G is C or N;

the dashed line represents the presence or absence of a bond;

m is 0-4; and

n and p are each independently 0-5.

Some embodiments of the present invention include:
1. A method of treating a skin wound or scar comprising administering to a subject in need thereof a composition comprising a therapeutically effective amount of a compound or a pharmaceutically acceptable salt thereof having a structure:

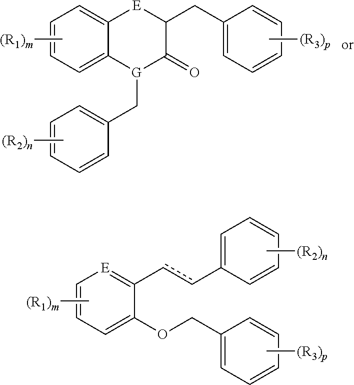

wherein:

R₁, R₂, and R₃ are each independently alkyl, alkoxy, halogen, trifluoromethyl, or —CO₂R₄, wherein R₄ is —H, C₁-C₆ alkyl, hydroxyalkyl, or —CH₂CH₂OH; or

each R₂ is independently

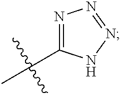 [SMILES: *c1nnn[nH]1]

acylsulfonamide, phosphonate, or —SO₃H;

each E is independently C, N, S, or O;

G is C or N;

the dashed line represents the presence or absence of a bond;

m is 0-4; and

n and p are each independently 0-5.

2. The method of embodiment 1, wherein the compound is selected from the group consisting of:

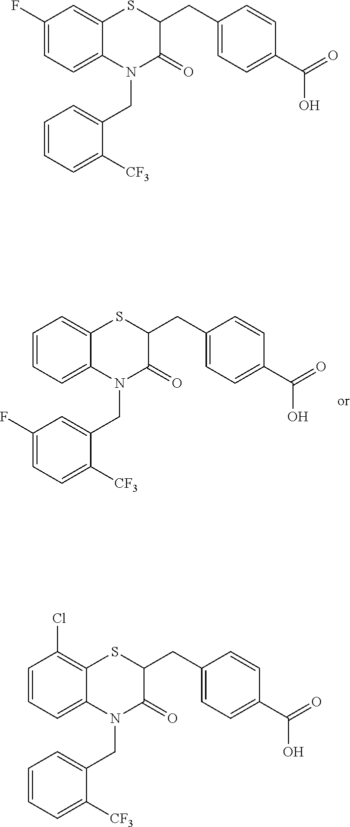 [SMILES: O=C(O)c1ccc(CC2Sc3cc(F)ccc3N(Cc3ccccc3C(F)(F)F)C2=O)cc1 ; O=C(O)c1ccc(CC2Sc3ccccc3N(Cc3cc(F)ccc3C(F)(F)F)C2=O)cc1 ; O=C(O)c1ccc(CC2Sc3c(Cl)cccc3N(Cc3ccccc3C(F)(F)F)C2=O)cc1]

and pharmaceutically acceptable salts, thereof.

3. The method of embodiment 1, wherein the compound is:

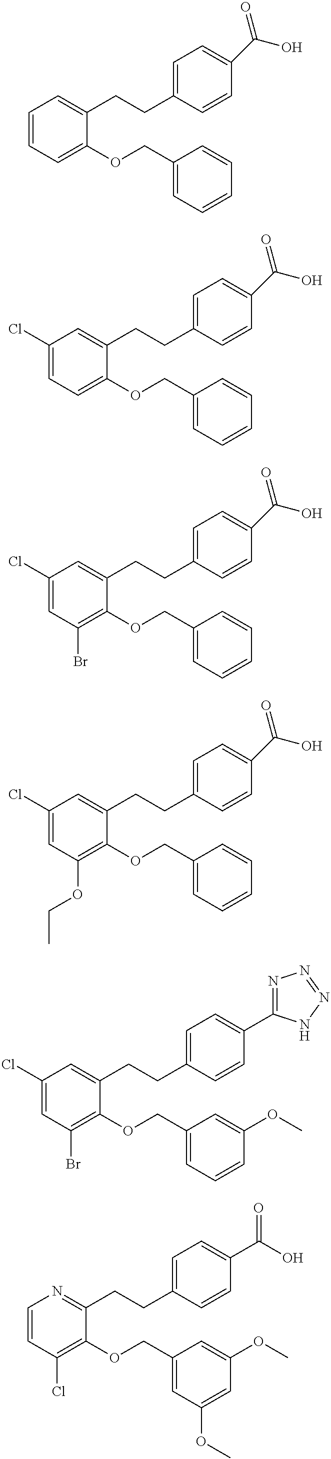

and pharmaceutically acceptable salts, thereof.

4. The method of embodiments 1-3, wherein the composition is administered by one selected from the group consisting of subcutaneously, subdermally, transdermally, intradermally or topically.

5. The method of embodiments 1-5, wherein the administration reduces formation of a scar type selected from the group consisting of at least hypertrophic scar, recessed scar, stretch mark, and/or a combination thereof.

6. The method of embodiments 1-5, wherein the composition is administered to a region selected from the group consisting of a face, neck, hands, arms, torso, back, legs, and/or a combination thereof.

7. The method of embodiments 1-6, wherein the composition is administered at a time selected from the group consisting of prior to surgical incision, during surgery, post-operatively, and/or a combination thereof

8. The method of embodiments 1-7, wherein said administration minimizes scar formation that would otherwise result in the absence of treatment.

9. The method of embodiments 1-7, wherein said administration prevents scar formation.

10. The method of embodiment 1, wherein a cause of said skin wound is selected from the group consisting of an incision, a laceration, a thermal burn, a chemical burn, an abrasion, a puncture wound, and a combination thereof.

11. The method of embodiments 1-10, wherein the composition is administered topically to the skin wound or scar and the tissue surrounding the skin wound or scar.

12. The method of embodiments 1-10, wherein the composition is injected into a skin wound or scar and the tissue surrounding the skin wound or scar.

13. The method of embodiments 1-11, wherein the composition is applied topically at least once a day.

14. The method of embodiments 1-13, wherein the composition is in the form of one selected from the group consisting of creams, suspensions, emulsions, lotions, gels, jellies, aqueous solutions, pastes, aerosols and sprays.

15. A method of treating, preventing or inhibiting wrinkle formation, comprising administering to a subject in need thereof a composition comprising a therapeutically effective amount of a compound or a pharmaceutically acceptable salt thereof having a structure:

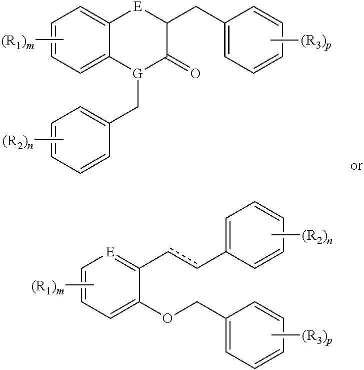

wherein:

R₁, R₂, and R₃ are each independently alkyl, alkoxy, halogen, trifluoromethyl, or —CO₂R₄, wherein R₄ is —H, C₁-C₆ alkyl, hydroxyalkyl, or —CH₂CH₂OH; or

each R₂ is independently

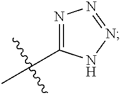 [SMILES: *c1nnn[nH]1]

acylsulfonamide, phosphonate, or —SO₃H;

each E is independently C, N, S, or O;

G is C or N;

the dashed line represents the presence or absence of a bond;

m is 0-4; and

n and p are each independently 0-5.

16. The method of embodiment 15, wherein the compound is selected from the group consisting of:

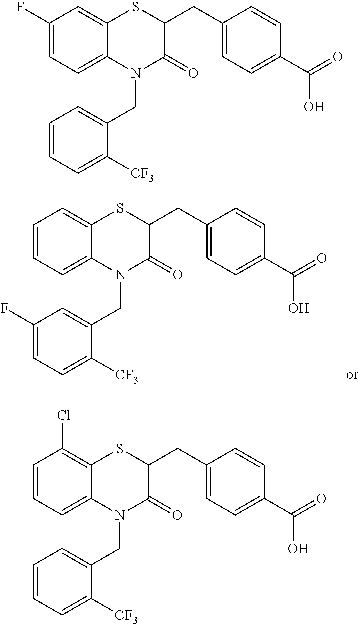

and pharmaceutically acceptable salts, thereof.

17. The method of embodiment 15, wherein the compound is selected from the group consisting of:

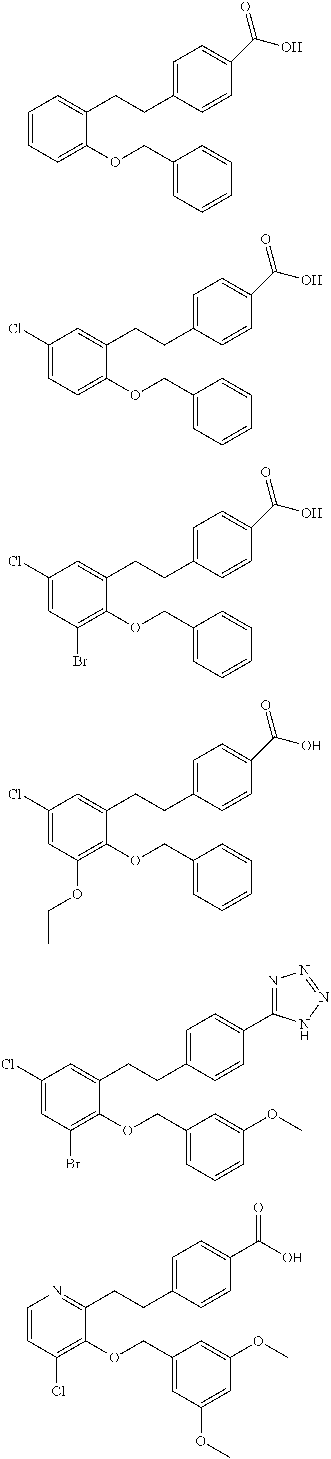

and pharmaceutically acceptable salts, thereof.

18. The method of embodiment 15, wherein the composition is administered topically to the skin wound or scar and the tissue surrounding the skin wound or scar.

19. The method of embodiment 15, wherein the composition is injected into a skin wound or scar and the tissue surrounding the skin wound or scar.

20. The method of embodiment 15, wherein the composition is applied topically at least once a day.

### DETAILED DESCRIPTION OF THE INVENTION

Disclosed herein are compositions and methods for wound healing and scar reduction.

In one embodiment of the invention there are provided methods of treating a skin wound or scar. Such methods are performed, for example, by administering to a subject in need thereof a composition comprising a therapeutically effective amount of a compound having a structure:

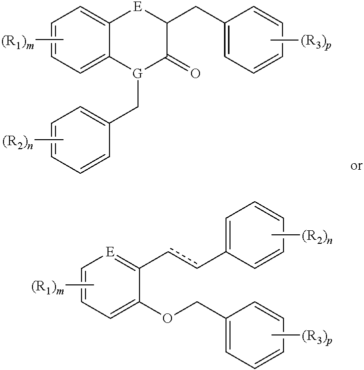

wherein:

R₁, R₂, and R₃ are each independently alkyl, alkoxy, halogen, trifluoromethyl, or —CO₂R₄, wherein R₄ is —H, C₁-C₆ alkyl, hydroxyalkyl, or —CH₂CH₂OH; or

each R₂ is independently

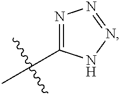 [SMILES: *c1nnn[nH]1]

acylsulfonamide, phosphonate, or —SO₃H

each E is independently C, N, S or O;

G is C or N;

the dashed line represents the presence or absence of a bond;

m is 0-4; and

n and p are each independently 0-5.

Exemplary compounds contemplated for use in the methods of the invention include, but are not limited to,

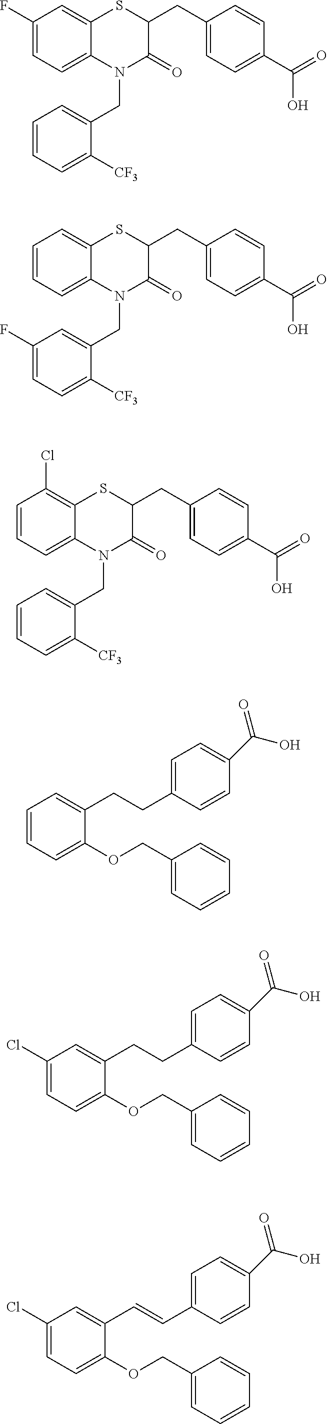 [SMILES: O=C(O)c1ccc(CC2Sc3cc(F)ccc3N(Cc3ccccc3C(F)(F)F)C2=O)cc1 ; O=C(O)c1ccc(CC2Sc3ccccc3N(Cc3cc(F)ccc3C(F)(F)F)C2=O)cc1 ; O=C(O)c1ccc(CC2Sc3c(Cl)cccc3N(Cc3ccccc3C(F)(F)F)C2=O)cc1 ; O=C(O)c1ccc(CCc2ccccc2OCc2ccccc2)cc1 ; O=C(O)c1ccc(CCc2cc(Cl)ccc2OCc2ccccc2)cc1 ; O=C(O)c1ccc(/C=C/c2cc(Cl)ccc2OCc2ccccc2)cc1]

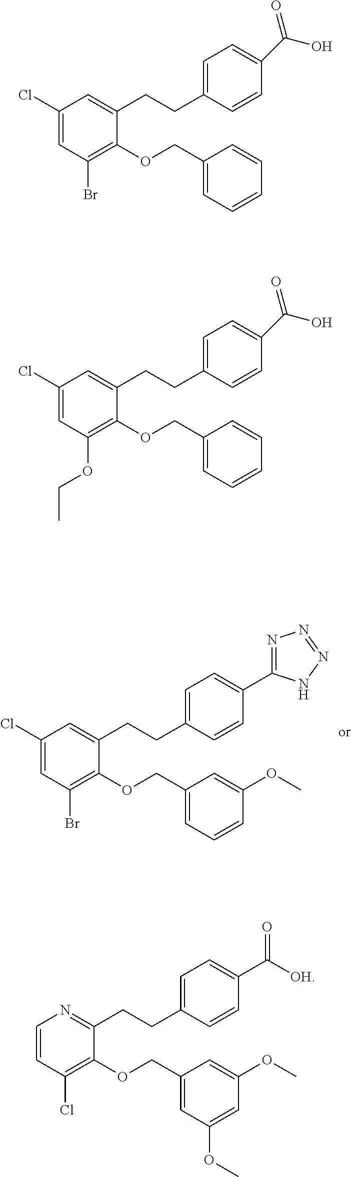 [SMILES: O=C(O)c1ccc(CCc2cc(Cl)cc(Br)c2OCc2ccccc2)cc1 ; CCOc1cc(Cl)cc(CCc2ccc(C(=O)O)cc2)c1OCc1ccccc1 ; COc1cccc(COc2c(Br)cc(Cl)cc2CCc2ccc(-c3nnn[nH]3)cc2)c1 ; COc1cc(COc2c(Cl)ccnc2CCc2ccc(C(=O)O)cc2)cc(OC)c1]

As used herein, the term “skin blemish” includes a flesh wound, scar, or wrinkle on any region of the skin of a body.

A “flesh wound” can be any area in which the structural integrity of the exterior surface of the skin is compromised. A flesh wound can be due to incision, laceration, abrasion, thermal burn, chemical burn, radiation or puncture of the skin. The wound can be superficial or extend to the deeper layers of the dermis, subcutaneous, deep fascia, muscle, bone or other internal organs.

A “scar” is an area of fibrous tissue (fibrosis) that replaces normal skin (or other tissue) after injury or disease. Scar types include hypertrophic scars, recessed scars, and stretch marks. Hypertrophic scars occur when the body overproduces collagen, which causes the scar to be raised above the surrounding skin. An example of a hypertrophic scar is a keloid scar. Atrophic, or recessed scars, have a sunken appearance and result when underlying support structure in the skin is lost. Stretch marks (striae) occur when skin is stretched rapidly (i.e., due to significant weight gain or growth spurt), or when skin is put under tension during the healing process, typically near a joint. As used herein, the term “scar” encompasses any type of scar in the skin due to any cause.

As used herein, the term “wrinkle” is a fold, ridge, crease, furrow, pit, crater, or sunken area in the skin that can be caused by habitual facial expressions, loss of collagen and/or elasticity due to aging, sun damage, smoking, poor hydration, and various other factors. A wrinkle can range from a deep crease to a fine line. Wrinkles occurring on any part of a body, in particular, wrinkles on head or neck of a subject are contemplated herein. Wrinkles that can be treated in accordance with the disclosure include, but are not limited to, a brow furrow, crows feet, nasolabial fold, one or more lines under the eyes or between the eye brows, and combinations thereof.

As used herein, “treatment” means to alleviate (or to eliminate) one or more features of a skin blemish either temporarily or permanently. When the compositions are administered to treat a wound, the compositions promote normal healing compared to a wound without the administration. That is, the size (length, depth, height and/or width), character, color and/or texture of the treated wound more closely resemble normal, non-wounded tissue. In this regard, treatment of a wound with the disclosed compositions can prevent, minimize or improve the appearance of a scar formation resulting from healing of the wound. Further, when the disclosed compositions are administered to treat a wrinkle, the wrinkle is treated if the appearance or prominence of the wrinkle is visibly or clinically diminished. That is the length and/or depth is decreased compared to the wrinkle prior to treatment. Alternatively, treatment can comprise prevention of a wrinkle. In this regard, the disclosed compositions can be applied to a region of the skin that typically develops a wrinkle, such as a forehead, lips, eyelids, nasolabial fold, skin under an eye, or between the eye brows in order to prevent the development of a wrinkle.

The disclosed compositions can be administered to prevent scar formation not associated with a wound, such as a stretch mark, or scars resulting from acne, chicken pox, measles or other disease states. In certain embodiments, the disclosed compositions are administered to the area of skin expansion in order to prevent formation of such scars. In these embodiments, the composition can be administered to any region of a face, abdomen, breasts, arms, legs, buttocks, back, or any other area where the skin is susceptible to developing a scar.

The compositions can be administered prior to, concurrently with, and/or after the development of the skin blemish. For instance, the disclosed compositions can be administered prior to an incision, during a surgical procedure, and/or any time post-operatively, and then additionally administered after the procedure as the healing process occurs. In another example, the compositions can be administered during pregnancy to prevent stretch marks. Alternately, the compositions can be administered after the development of a blemish. Also, compositions can be administered in conjunction with scar reduction treatments such as surgery, laser resurfacing and dermabrasion.

The compositions may be administered between 1 and 7 days a week, for a period of time necessary to achieve the desired results, which may be several days to several months. The compositions can be administered once or several times (2, 3, 4, or more times) a day depending on the desired effect. In certain embodiments, the compositions can be administered every 1, 2, 3, 4, 5, 6, or 7 days. In another embodiment, the compositions can be administered one or more times every 1, 2, 3, or 4 weeks. The administration can be on a monthly or bi-monthly basis. Further, the compositions can be administered for 1, 2, 3, 6, 9, or 12 months or more. In certain embodiments, the compositions can be administered on an ongoing basis to maintain a desired result.

The disclosed compounds can be administered as part of a composition. As used herein, “formulation” and “composition” may be used interchangeably and refer to a combination of elements that is presented together for a given purpose. Such terms are well known to those of ordinary skill in the art.

As used herein, “carrier,” “inert carrier,” and “acceptable carrier” may be used interchangeably and refer to a carrier which may be combined with the presently disclosed compounds in order to provide a desired composition. Those of ordinary skill in the art will recognize a number of carriers that are well known for making specific pharmaceutical and/or cosmetic compositions. Desirably, the carrier is suitable for application to keratinous surfaces or other areas of the body. Upon application, acceptable carriers are substantially free of adverse reactions with skin and other keratinous surfaces. For example, the carriers may take the form of fatty or non-fatty creams, milky suspensions or emulsion-in-oil or oil-in-water types, lotions, gels or jellies, colloidal or non-colloidal aqueous or oily solutions, pastes, aerosols, soluble tablets or sticks. In accordance with one embodiment, the composition includes a dermatologically compatible vehicle or carrier. The vehicle which may be employed for preparing compositions may comprise, for example, aqueous solutions such as e.g., physiological salines, oil solutions or ointments. The vehicle furthermore may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid; these may be used for increasing the viscosity.

The compounds of the present invention can form pharmaceutically acceptable salts which are also within the scope of this invention. Reference to a compound is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds may be formed, for example, by reacting a compound with an amount of acid or base, such as an equivalent amount, in a medium such as one in which the salt precipitates or in an aqueous medium followed by lyophilization. Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

Examples of additional agents which can be included in the present compositions are anti-itch, anti-cellulite, anti-scarring, and anti-inflammatory agents, anesthetics, anti-irritants, vasoconstrictors, vasodilators, as well as agents to prevent/stop bleeding, and improve/remove pigmentation, moisturizers, desquamating agents, tensioning agents, anti-acne agents. Anti-itch agents can include methyl sulphonyl methane, sodium bicarbonate, calamine, allantoin, kaolin, peppermint, tea tree oil and combinations thereof. Anti-cellulite agents can include forskolin, xanthine compounds such as, but not limited to, caffeine, theophylline, theobromine, and aminophylline, and combinations thereof. Anesthetic agents can include lidocaine, benzocaine, butamben, dibucaine, oxybuprocaine, pramoxine, proparacaine, proxymetacaine, tetracaine, and combinations thereof. Anti-scarring agents can include IFN-.gamma, fluorouracil, poly(lactic-co-glycolic acid), methylated polyethylene glycol, polylactic acid, polyethylene glycol and combinations thereof. Anti-inflammatory agents can include dexamethasone, prednisolone, corticosterone, budesonide, estrogen, sulfasalazine, mesalamine and derivatives and combinations thereof. Additionally, active agents such as epinephrine, thymidine, cytidine, uridine, antiypyrin, aminocaproic acid, tranexamic acid, eucalyptol, allantoin, glycerin, and sodium selenite, can be included. Formulations can further comprise degradation inhibitors. Degradation inhibitors, include but are not limited to, glycosaminoglycans (e.g., heparin, heparin sulfate, dermatan sulfate, chrondroitin sulfate, o-sulfated HA, lnamarin, and amygdalin), antioxidants (e.g. ascorbic acid, melatonin, vitamin C, vitamin E), proteins (e.g., serum hyaluronidase inhibitor), and fatty acids (e.g. saturated C₁₀ to C₂₂ fatty acids). In certain embodiments, additional active agent is an antioxidant. In certain embodiments, the antioxidant comprises a vitamin C and/or a vitamin E such as d-alpha-tocopheryl polyethylene glycol 1000 succinate (TPGS).

The disclosed compositions are well suited for topical, subcutaneous, intradermal, subdermal, subcutaneous, and trandermal administration. Topical administration relates to the use of a composition applied to the surface of the skin at the site of a skin blemish for exertion of local action. Accordingly, such topical compositions include those pharmaceutical or cosmetic forms in which the composition is applied externally by direct contact with the skin surface to be treated, such as the face, neck, arms, legs, and/or torso. Conventional pharmaceutical or cosmetic forms for this purpose include ointments, liniments, creams, shampoos, lotions, pastes, jellies, sprays, aerosols, and the like, and may further be applied directly or in patches or impregnated dressings depending on blemish and skin region to be treated. The term “ointment” embraces formulations (including creams) having oleaginous, water-soluble and emulsion-type bases, e.g., petrolatum, lanolin, polyethylene glycols, as well as mixtures of these.

The compositions are appropriate for mesotherapy applications as well. Mesotherapy is a non-surgical cosmetic treatment technique involving intra-epidermal, intra-dermal, and/or subcutaneous injection of a composition. The compositions are administered in the form of small multiple droplets into the epidermis, dermo-epidermal junction, and/or the dermis.

In accordance with the disclosure, a pharmaceutical or cosmetic composition can optionally include one or more agents such as, without limitation, emulsifying agents, wetting agents, sweetening or flavoring agents, tonicity adjusters, preservatives, buffers antioxidants and flavonoids. Tonicity adjustors useful in a pharmaceutical composition of the present disclosure include, but are not limited to, salts such as sodium acetate, sodium chloride, potassium chloride, mannitol or glycerin and other pharmaceutically acceptable tonicity adjusters. Preservatives useful in the pharmaceutical compositions described herein include, without limitation, benzalkonium chloride, chlorobutanol, thimerosal, phenyl mercuric acetate, and phenyl mercuric nitrate. Various buffers and means for adjusting pH can be used to prepare a pharmaceutical composition, including but not limited to, acetate buffers, citrate buffers, phosphate buffers and borate buffers. Similarly, antioxidants useful in pharmaceutical compositions are well known in the art and include for example, sodium metabisulfite, sodium thiosulfate, acetylcysteine, butylated hydroxyanisole and butylated hydroxytoluene. Flavonoids are compounds found in plants that are well known to have diverse beneficial biochemical and antioxidant effects. Subcategories of flavonoids include: flavones, flavonols, flavanonse and flavanonols. Examples of flavonoids include: luteolin, apigenin, tangeritin, quercetin, kaempferol, myricetin, fisetin, isorhamnetin, pachypodol, rhamnazin, hesperetin, naringenin, eriodictyol, homoeriodictyol, taxifolin, dihydroquercetin, dihydrokaempferol, tannic acid, tannis, condensed tannis, and hydrolysable tannis. It is understood that these and other substances known in the art can be included in a pharmaceutical or cosmetic composition disclosed herein.

As used herein, the term “therapeutically effective amount” means the amount of the pharmaceutical or cosmetic composition that will elicit the biological, medical, or cosmetic response of a subject in need thereof that is being sought by the researcher, veterinarian, medical doctor or other clinician. In some embodiments, the subject in need thereof is a mammal. In certain embodiments, the mammal is human. Effective amounts of the compound may be determined by one of ordinary skill in the art but will vary depending on the compound employed, frequency of application and desired result, and will generally range from about 0.0000001% to about 50%, by weight, of the composition, preferably from about 0.001% to about 50%, by weight, of total composition, more preferably from about 0.001% to about 30%, by weight of the composition.

The compounds described herein may be administered at least in the minimum dose necessary to achieve the desired therapeutic effect. Generally, such doses will be in the range of about 1 mg/day to about 1000 mg/day; more preferably in the range of about 10 mg/day to about 500 mg/day. In another example embodiment, the compound or compounds may be present in a composition or formulation in a range of about 0.0001 mg/kg/day to about 100 mg/kg/day or about 0.01 mg/kg/day to about 100 mg/kg/day. However, the actual amount of the compound to be administered in any given case will be determined by a physician taking into account the relevant circumstances, such as the age and weight of a patient, patient's general physical condition, severity of the skin blemish, and route of administration. In some instances, dosing is evaluated on a case-by-case basis. Compositions may be applied topically, by injection, applied in a transdermal delivery system and by microporation technology.

Additionally, compositions may be designed to delay release of the compound over a given period of time, or to carefully control the amount of compound released at a given time during the course of treatment.

The pH of the disclosed compositions can be about 3 to about 8.0, or about 6.5 to about 7.5. In certain embodiments, the pH of the formulation is about 7.0 to about 7.4 or about 7.1 to about 7.3.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Any reference made to patents and printed publications throughout this specification is individually incorporated herein by reference in its entirety.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### EXAMPLES

TABLE 1

| | EP₂ | EP₂ | EP₄ | EP₄ |
|---|---|---|---|---|
| | cAMP | Kᵢ | cAMP | Kᵢ |
| Compound | EC₅₀ (nM) | IC₅₀ (nM) | EC₅₀ (nM) | IC₅₀ (nM) |
| 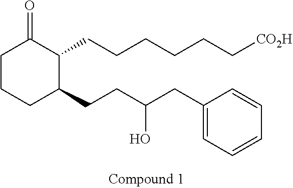 [SMILES: O=C(O)CCCCCC[C@H]1C(=O)CCC[C@@H]1CCC(O)Cc1ccccc1] | >10⁴ | >10⁴ | 0.9 | 81 |
| 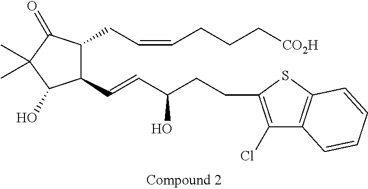 [SMILES: CC1(C)C(=O)[C@H](C/C=C\CCCC(=O)O)[C@@H](/C=C/[C@H](O)CCc2sc3ccccc3c2Cl)[C@@H]1O] | >10⁴ | >10⁴ | 0.3 | 7 |
| 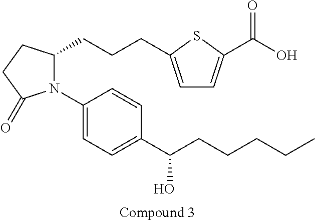 [SMILES: CCCCC[C@H](O)c1ccc(N2C(=O)CC[C@@H]2CCCc2ccc(C(=O)O)s2)cc1] | 0.19 | 21 | >10⁴ | >10⁴ |
| 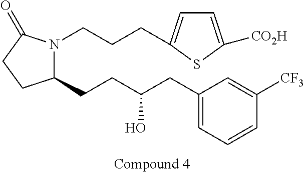 [SMILES: O=C(O)c1ccc(CCCN2C(=O)CC[C@@H]2CC[C@@H](O)Cc2cccc(C(F)(F)F)c2)s1] | 71 | 4647 | 0.08 | 2 |
| 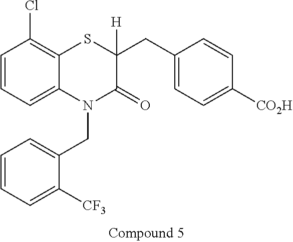 [SMILES: O=C(O)c1ccc(CC2Sc3c(Cl)cccc3N(Cc3ccccc3C(F)(F)F)C2=O)cc1] | 1.9 | 1.0 | 4 | 0.3 |

### Incisional Skin Wound Model and Assessment

Sprague-Dawley rats at 180-200 gram were anesthetized with isoflourane. After shaving, 2-cm long incisions were made on the left and right side of the back, reaching the deep fascia on the back skin of rats under sterile conditions. Incisional wounds were immediately closed with 4.0 sutures, and then topically treated with a vehicle or test drugs at 0.004% twice daily for 5 days. The vehicle used here contains ethanol 30%, propylene glycol 12%, dipropylene glycol 5%, benzyl alcohol 5%, glycerol 3% and normal saline 45%.

Wounds were photographed on day 7. All photos were coded and scored by lay people. Evaluation of wound sites was based on scar width, palpability (elevation) of wound areas, and general progress in healing, using a scale of 0 to 6; the severer a scar, the higher the score. Each scar was divided into 4 regions, separated by suture sites; each quarter was scored independently; the mean of the 4 part scores was recorded as the overall scar score of each incision site.

TABLE 2
Comparison of scar scores of incisional wounds on Day 7 in rats topically
treated with test drugs at 0.004% or vehicle for 5 days.

| | Compound 2 | | Compound 4 | | Compound 5 | |
|---|---|---|---|---|---|---|
| | Vehicle- | Drug- | Vehicle- | Drug- | Vehicle- | Drug- |
| | treated | treated | treated | treated | treated | treated |
| Scar Scores | 1.5 ± 0.3 | 0.60 ± 0.1 | 1.3 ± 0.07 | 0.76 ± 0.07 | 1.2 ± 0.07 | 0.56 ± 0.07 |

Although the invention has been described with reference to embodiments and examples, it should be understood that numerous and various modifications can be made without departing from the spirit of the invention. Accordingly, the invention is limited only by the following claims.

What is claimed is:

1. A method of treating a skin wound or scar comprising administering to a subject in need thereof a composition comprising a therapeutically effective amount of a compound or a pharmaceutically acceptable salt thereof having a structure: 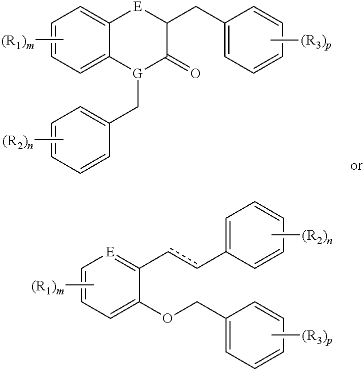
wherein:
R₁, R₂, and R₃ are each independently alkyl, alkoxy, halogen, trifluoromethyl, or —CO₂R₄, wherein R₄ is —H, C₁-C₆ alkyl, hydroxyalkyl, or —CH₂CH₂OH; or
each R₂ is independently 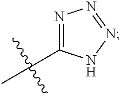 [SMILES: *c1nnn[nH]1]
acylsulfonamide, phosphonate, or —SO₃H;
each E is independently C, N, S, or O;
G is C or N;
the dashed line represents the presence or absence of a bond;
m is 0-4; and
n and p are each independently 0-5.

2. The method of claim 1, wherein the compound is: 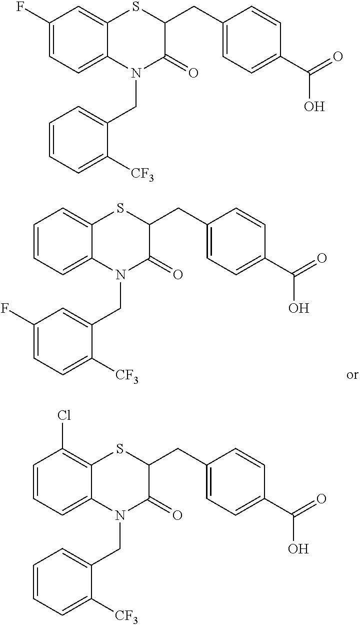
and pharmaceutically acceptable salts, thereof.

3. The method of claim 1, wherein the compound is: 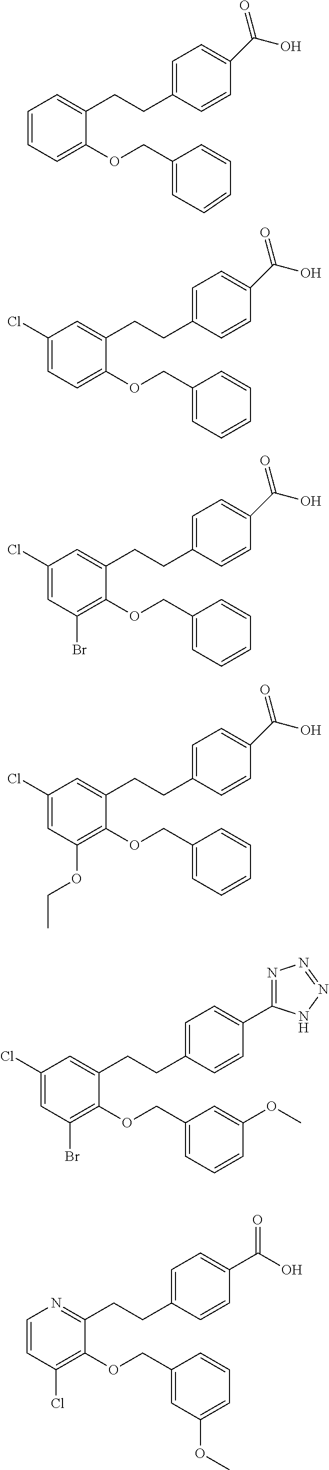
and pharmaceutically acceptable salts, thereof.

4. The method of claim 1, wherein the composition is administered subcutaneously, subdermally, transdermally, intradermally or topically.

5. The method of claim 1, wherein the administration reduces formation of a scar type selected from the group consisting of hypertrophic scar, recessed scar, stretch mark, and a combination thereof.

6. The method of claim 1, wherein the composition is administered to a region selected from the group consisting of a face, neck, arms, hands, torso, back, legs, and a combination thereof.

7. The method of claim 1, wherein the composition is administered at a time selected from the group consisting of prior to surgical incision, during surgery, post-operatively, and a combination thereof.

8. The method of claim 1, wherein said administration minimizes scar formation.

9. The method of claim 1, wherein said administration prevents scar formation.

10. The method of claim 1, wherein a cause of said skin wound is selected from the group consisting of an incision, a laceration, a thermal burn, a chemical burn, an abrasion, a puncture wound, and a combination thereof.

11. The method of claim 1, wherein the composition is administered topically to the skin wound or scar and the tissue surrounding the skin wound or scar.

12. The method of claim 1 wherein the composition is injected into a skin wound or scar and the tissue surrounding the skin wound or scar.

13. The method of claim 1, wherein the composition is applied topically at least once a day.

14. The method of claim 1, wherein the composition is in the form of one selected from the group consisting of creams, suspensions, emulsions, lotions, gels, jellies, aqueous solutions, pastes, aerosols and sprays.

15. A method of treating, preventing or inhibiting wrinkles, comprising administering to a subject in need thereof a composition comprising a therapeutically effective amount of a compound or a pharmaceutically acceptable salt thereof having a structure: 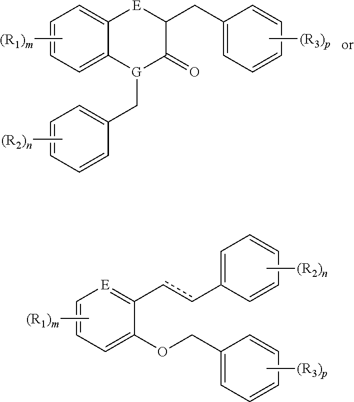
wherein:
R₁, R₂, and R₃ are each independently alkyl, alkoxy, halogen, trifluoromethyl, or —CO₂R₄, wherein R₄ is —H, C₁-C₆ alkyl, hydroxyalkyl, or —CH₂CH₂OH; or
each R₂ is independently 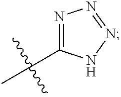 [SMILES: *c1nnn[nH]1]
acylsulfonamide, phosphonate, or —SO₃H;
each E is independently C, N, S, or O;
G is C or N;
the dashed line represents the presence or absence of a bond;
m is 0-4; and
n and p are each independently 0-5.

16. The method of claim 15, wherein the compound is: 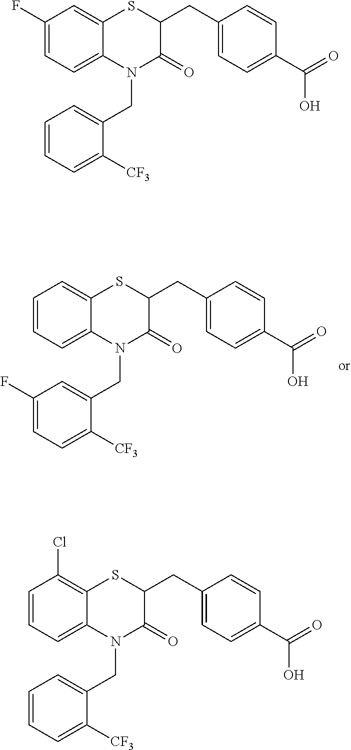 [SMILES: O=C(O)c1ccc(CC2Sc3cc(F)ccc3N(Cc3ccccc3C(F)(F)F)C2=O)cc1 ; O=C(O)c1ccc(CC2Sc3ccccc3N(Cc3cc(F)ccc3C(F)(F)F)C2=O)cc1 ; O=C(O)c1ccc(CC2Sc3c(Cl)cccc3N(Cc3ccccc3C(F)(F)F)C2=O)cc1]
and pharmaceutically acceptable salts, thereof.

17. The method of claim 15, wherein the compound is selected from the group consisting of: 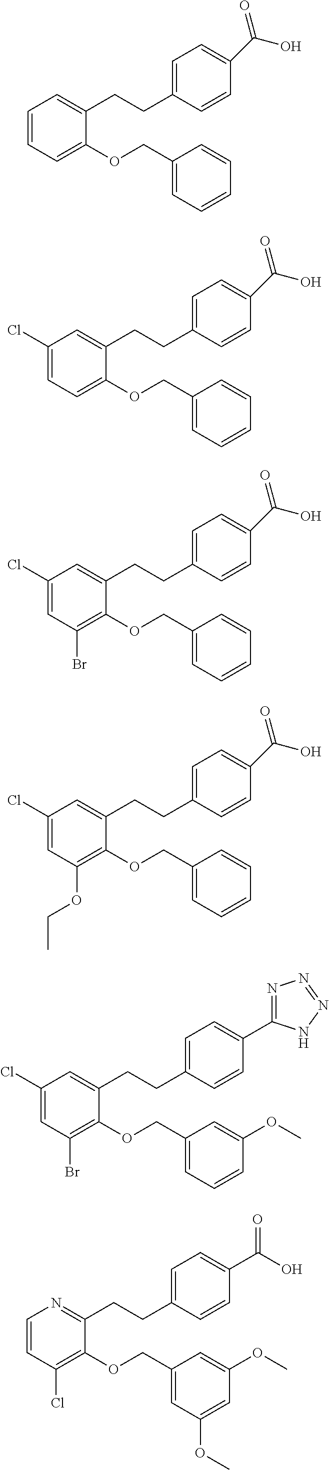 [SMILES: O=C(O)c1ccc(CCc2ccccc2OCc2ccccc2)cc1 ; O=C(O)c1ccc(CCc2cc(Cl)ccc2OCc2ccccc2)cc1 ; O=C(O)c1ccc(CCc2cc(Cl)cc(Br)c2OCc2ccccc2)cc1 ; CCOc1cc(Cl)cc(CCc2ccc(C(=O)O)cc2)c1OCc1ccccc1 ; COc1cccc(COc2c(Br)cc(Cl)cc2CCc2ccc(-c3nnn[nH]3)cc2)c1 ; COc1cc(COc2c(Cl)ccnc2CCc2ccc(C(=O)O)cc2)cc(OC)c1]
and pharmaceutically acceptable salts, thereof.

18. The method of claim 15, wherein the composition is administered topically to the skin wound or scar and the tissue surrounding the skin wound or scar.

19. The method of claim 15, wherein the composition is injected into a skin wound or scar and the tissue surrounding the skin wound or scar.

20. The method of claim 15, wherein the composition is applied topically at least once a day.
